(19) United States
(12) Patent Application Publication
(10) Pub. No.: US 2009/0299122 A1
(43) Pub. Date: Dec. 3, 2009

(54) PROCESS FOR PRODUCING A PURIFIED HYDROCARBON GAS

(76) Inventors: Henricus Abraham GEERS, Rijswijk (NL); Esther Lucia Johanna Van Soest-Vercammen, Amsterdam (NL)

Correspondence Address: SHELL OIL COMPANY, P O BOX 2463, HOUSTON, TX, 772522463, US

(21) Appl. No.: 12/473,523

(22) Filed: May 28, 2009

(30) Foreign Application Priority Data
May 30, 2008 (EP) 08157278.6

(51) Int. Cl. C07C 7/11

(52) U.S. Cl. 585/857; 585/833; 585/860; 585/862; 585/864; 585/866

(57) ABSTRACT

Process for producing purified hydrocarbon gas from a gas stream comprising methane and acidic contaminants, which process comprises the steps cooling the gas stream by expansion to form a mixture comprising solid and/or liquid acidic contaminants and a vapour containing gaseous hydrocarbons and a reduced amount of acidic contaminants; separating the solid and/or liquid acidic contaminants from the first mixture, yielding partly purified gas; compressing the partly purified gas; and contacting the compressed partly purified gas with an absorbing liquid to yield the purified hydrocarbon gas.

### RELATED APPLICATIONS

The present application claims priority to co-pending European Patent Application number 08157278.6-1213, filed on May 30, 2008, and having attorney docket number TS6940 EPC. European Patent Application number 08157277.8 is herein incorporated by reference in its entirety.

### FIELD OF THE INVENTION

The present invention relates to a process for producing purified hydrocarbon gas. The invention especially relates to producing purified hydrocarbon gas from natural gas that contains carbon dioxide and optionally hydrogen sulphide.

### BACKGROUND OF THE INVENTION

Such a process is known from WO-A 2004/070297. This document discloses a process in which a natural gas stream comprising methane and acidic contaminants is first cooled to remove water from the natural gas, and subsequently the natural gas is further cooled to solidify acidic contaminants or dissolve such contaminants in a liquid, which contaminants are removed so that a purified natural gas is recovered.

It has been found that this process is very suitable when the natural gas stream contains relatively small amounts of acidic contaminants, such as up to 25% vol. However, there is room for improvement of this process when the natural gas streams contain high concentrations, i.e. at least 25 volume %, of acidic contaminants.

A two step process is known from WO-A 2007/030888, which document discloses a process in which a natural gas stream comprising methane and acidic species is dehydrated and subsequently cooled by expansion to obtain a slurry of solid acidic contaminants and liquid hydrocarbons together with a gaseous stream containing gaseous acidic species. The slurry is removed and the gaseous stream containing the gaseous acidic species is treated with a solvent, e.g., methanol, to wash the gaseous acidic species from the gaseous stream, resulting in a purified natural gas product. The acidic species are contained in the solvent, and are recovered from the solvent in a subsequent desorption step. The solvent may be recycled to the wash treatment after a number of heat exchange steps.

This process has the disadvantage that the gas has been expanded so that a large volume of gas has to be treated in the wash step. Moreover, a solvent such as methanol also dissolves an amount of hydrocarbons which results in a loss of valuable product in the purified natural gas product.

### SUMMARY OF THE INVENTION

It has now been found that an efficient removal of acidic contaminants from gases such as natural gas with a high content of acidic contaminants can be obtained without incurring the risk of significant losses of hydrocarbon product.

Accordingly, the present invention provides a process for producing purified hydrocarbon gas from a gas stream comprising methane and acidic contaminants, which process comprises the steps:

(a) cooling the gas stream by expansion to form a mixture comprising solid and/or liquid acidic contaminants and a vapour containing gaseous hydrocarbons and a reduced amount of acidic contaminants;
(b) separating the solid and/or liquid acidic contaminants from the first mixture, yielding partly purified gas;
(c) compressing the partly purified gas; and
(d) contacting the compressed partly purified gas with an absorbing liquid to yield the purified hydrocarbon gas.

### BRIEF DESCRIPTION OF THE FIGURES

FIG. 1 illustrates a natural gas stream that may be treated in the process according to the present invention.

### DETAILED DESCRIPTION OF THE INVENTION

The present process provides a solution to the purification of gas streams that contain relatively large amounts of acidic contaminants. In the first stage a large proportion of the acidic contaminants are solidified and/or liquefied and the thus formed solids and/or liquids are subsequently removed, whereas the partly purified gas contains the gaseous hydrocarbons and a reduced amount of vaporous acidic contaminants. Because a substantial amount of acidic contaminants, representing a potentially significant portion of the gas stream, has been removed in the separation stage, a smaller amount of gas needs to be compressed in the further steps of the process. Hence a smaller volume of partly purified gas is contacted with the absorbing liquid. Since the absorbing liquid contains one or more amine compounds, the solution will absorb virtually no hydrocarbons, whereas the absorption capacity of the solution for acidic contaminants is excellent.

The gas stream can be any stream of gas that comprises acidic contaminants and hydrocarbons. In particular the process according to the present invention can be applied to a natural gas stream, i.e., a gas stream that contains significant amounts of methane and that has been produced from a subsurface reservoir. It includes a methane natural gas stream, an associated gas stream or a coal bed methane stream. The amount of the hydrocarbon fraction in such a gas stream is suitably from 10 to 85 mol % of the gas stream, preferably from 25 to 75 mol %. Especially the hydrocarbon fraction of the natural gas stream comprises at least 75 mol % of methane, preferably 90 mol %. The hydrocarbon fraction in the natural gas stream may suitably contain from 0 to 20 mol %, suitably from 0.1 to 10 mol %, of C₂-C₆ compounds. The gas stream may also comprise up to 20 mol %, suitably from 0.1 to 10 mol % of nitrogen, based on the total gas stream.

In the process of the invention the acidic contaminants are in particular carbon dioxide and/or hydrogen sulphide. It is observed that also minor amounts of other contaminants may be present, e.g. carbon oxysulphide, mercaptans, alkyl sulphides and aromatic sulphur-containing compounds. The major part of these components will also be removed in the process of the present invention.

The amount of hydrogen sulphide in the gas stream containing methane is suitably in the range of from 5 to 40 volume % of the gas stream, preferably from 20 to 35 volume % and/or the amount of carbon dioxide is in the range of from 10 to 90 vol %, preferably from 20 to 75 vol %, based on the total gas stream. It is observed that the present process is especially suitable for gas streams comprising large amounts of contaminants, e.g. 10 vol % or more, suitably between 15 and 90 vol %.

Gas stream containing the large amounts of contaminants as described above cannot be processed using conventional techniques as amine extraction techniques as they will become extremely expensive, especially due to the large amounts of heat needed for the regeneration of loaded amine solvent.

As indicated above, acidic contaminants that are usually present in natural gas streams include hydrogen sulphide and carbon dioxide. It is also possible that a natural gas stream contains other components, including ethane, propane and hydrocarbons with four or more carbon atoms. It will be appreciated that when a portion of acidic contaminants, e.g., carbon dioxide, solidifies and/or liquefies in the cooling stages, other components, e.g., hydrogen sulphide and hydrocarbons, may liquefy. The liquid components are suitably removed together with the solid and/or liquid acidic contaminants from the vapour.

The gas stream, and in particular natural gas streams produced from a subsurface formation, may typically contain water. In order to prevent the formation of gas hydrates in the present process, at least part of the water is suitably removed. Therefore, the gas stream that is used in the present process has preferably been dehydrated. This can be done by conventional processes. A suitable process is the one described in WO-A 2004/070297. Other processes for forming methane hydrates or drying natural gas are also possible. Other dehydration processes are also possible, including treatment with molecular sieves or drying processes with glycol or methanol. Suitably, water is removed until the amount of water in the gas stream comprises at most 50 ppmw, preferably at most 20 ppmw, more preferably at most 1 ppmw of water, based on the total gas stream.

In a first step of the present process the gas stream is cooled via expansion. Preferably the expansion is done by isenthalpic expansion, preferably isenthalpic expansion over an orifice or a valve, especially a Joule-Thomson valve or a series of Joule-Thomson valves. In another preferred embodiment the expansion is done by nearly isentropic expansion, especially by means of an expander, preferably a turbo expander, or a laval nozzle. The skilled person will appreciate that expansion causes a lowering of temperature, so that cooling may be achieved by expansion and adapting pressure. In the present process it is preferred to use the energy for cooling as efficiently as possible. Therefore, the cooling stage preferably comprises, in addition, one or more heat exchange steps. The heat exchange steps may be indirect heat exchange or direct heat exchange, e.g., by spraying with a cold liquid, as shown in WO-A 2004/070297. Preferably, the gas stream is subjected to indirect heat exchange with one or more cold process streams or external streams. Cold external streams may be suitable streams from an LNG (liquefied natural gas) plant, such a cold LNG stream or a refrigerant stream, or from an air separation unit. A suitable heat exchange step is between the gas stream and the partly purified gas exiting the cooling stage. Another suitable heat exchange can be effected between the gas stream and the solid and/or liquid acidic contaminants that are separated from the aforesaid vapour.

Gas streams, such as natural gas streams, may become available at a temperature of −5 to 150° C. and a pressure of 10 to 700 bar, suitably from 20 to 200 bar. Although indirect heat exchange may be effective to accomplish significant cooling of the gas stream, the cooling stage comprises one or more expansion steps. These expansion steps may be done via a Joule-Thomson valve, a venturi tube or a turbo-expander or any other expansion means known in the art.

As indicated above, the cooling eventually leads to liquid and preferably solid acidic contaminants. It is preferred to achieve the cooling in several steps, e.g., by indirect heat exchange and expansion. It is also possible to solidify by spraying with a cold liquid, as shown in WO-A 2004/070297. Suitably, solid and/or liquid acidic contaminants are obtained in a final expansion step. The final expansion step is preferably achieved over a Joule-Thomson valve. Therefore, preferably, in a first step, which may be achieved by various intermediate steps and various methods, the gas stream is cooled to a temperature ranging from 1 to 40° C. above the freeze out temperature of the first acidic contaminant to freeze out, the freeze out temperature being the temperature at which solid contaminants are formed. Preferably, the cooling is effected till from 2 to 10° C. above the freeze out temperature. In a final step the gas stream is preferably cooled to the temperature at which a mixture of solid and/or liquid acidic contaminants and a vapour comprising gaseous hydrocarbons are formed by expansion over a valve. Preferably, the gas stream is partly or completely liquid before being expanded over the valve, and solid contaminants are formed upon expansion. This ensures a better separation performance in the vessel. Suitably, the gas stream is expanded from a pressure ranging from 20 to 200 bar to a pressure of 10 to 40 bar. Expansion over this pressure range suitably causes that liquid and/or solid acidic contaminants are formed.

The liquefaction and/or solidification of acidic contaminants may take place very rapidly, especially upon expansion over a valve, thereby forming the first mixture. To facilitate the separation the mixture is passed into a vessel, wherein the separation between solid and/or liquid acidic contaminants and vapour can take place. By gravity the solid acidic contaminants, and any other liquid that is formed, drops to the bottom of the vessel. After such separation the solid and/or liquid acidic contaminants are removed from the process. Since it is easier to transport liquids than to transport solids, it is preferred to melt at least partly the solid acidic contaminants, if formed. Such melting can be accomplished by heating the solids in the vessel by means of an electric immersion heater, by a bundle coil, i.e., a type of indirect heat exchanger, through which a process stream is fed, or by injecting a relatively warm fluid, such as a condensate. These measures have been suggested in WO-A 2004/0702897 and WO-A 2007/030888. In the present process it is preferred to heat at least a part of the withdrawn contaminants in a liquid, solid or slurry phase, and recycle at least a part of thus heated contaminants, in liquid or gaseous phase, to the vessel. In this way no extraneous material is recycled to the vessel. Preferably, all solid acidic contaminants are melted. In this way a liquid stream of contaminants is obtained, which can be easily transported further.

In a preferred embodiment, step (b) is performed in a separation vessel and comprises the steps of:

(b1) introducing a stream comprising liquid acidic contaminants into the intermediate or the bottom part or both of the separation vessel to obtain a diluted slurry of acidic contaminants;
(b2) introducing the diluted slurry of acidic contaminants via a slurry pump, preferably an eductor, into a heat exchanger in which solid acidic contaminant present in the diluted slurry of contaminants is melted into liquid acidic contaminant, wherein the heat exchanger is positioned outside the separation vessel, and the slurry pump, preferably the eductor, is arranged inside or outside the separation device or partly inside and outside the separation vessel;
(b3) introducing part or all of the liquid contaminant obtained in step b2 into a gas-liquid separator, wherein the gas-liquid separator is preferably the bottom part of the separation vessel;
(d4) introducing part or all of the liquid contaminant obtained in step b3 into the separation vessel as described above;
(b5) removing from the gas-liquid separator a stream of liquid acidic contaminant; and optionally
(b6) separating the stream of liquid contaminant obtained in step b5 into a liquid product stream and a recirculation stream which is used as a motive fluid in the eductor in the case that an eductor is used.

In this preferred embodiment, a continuously moving slurry phase is obtained, minimizing the risk of any blockages in the cryogenic separation vessel or in the pipelines and other pieces of equipment. Further, when a fully liquid stream is withdrawn from the heat exchanger, the absence of solid contaminant reduces the risk of blockages or erosion in subsequent pipelines or other equipment, and no damages will occur to any devices having moving parts, such as pumps. Moreover, when a pure liquid stream is withdrawn from the heat exchanger, a relatively cold liquid stream is obtained, thus minimizing the heat requirements of the separation device, and maintaining a high amount of exchangeable cold in the product stream.

In the event that the contaminant-rich stream mainly comprises carbon dioxide and is therefore a CO₂-rich stream, preferably CO₂-rich stream is further pressurised and injected into a subterranean formation, preferably for use in enhanced oil recovery or for storage into an aquifer reservoir or for storage into an empty oil reservoir. It is an advantage that a liquid CO₂-rich stream is obtained, as this liquid stream requires less compression equipment to be injected into a subterranean formation.

The partly purified gas becomes available at a reduced pressure. Before contacting the partly purified gas to the absorbing liquid comprising one or more amine compounds, the partly purified gas is compressed. Such compressing can be done after heat exchange, e.g., with the gas stream as indicated above. Preferably, the partly purified gas is compressed in one or more compression steps. In order to make optimal use of the energy that is released at an earlier expansion step, the energy that is recovered at such expansion step or steps of the natural gas stream is preferably used for the compression step or steps of the partly purified gas. Since the volume of partly purified gas is smaller than that of the natural gas stream the expansion energy can compensate at least a significant part of the required compression energy.

The partly purified gas is preferably brought to a temperature ranging at least 1° C., preferably at least 2° C., more preferably at least 20° C., even more preferably at least 40 above the freeze out temperature of the first acidic contaminant to freeze out, the freeze out temperature being the temperature at which solid contaminants are formed. As indicated above, the freeze out temperature also depends on the prevailing pressure. Hence, if the partly purified gas has been reheated, e.g., by heat exchange with the gas stream or due to the compression, cooling may be appropriate, e.g., by means of indirect heat exchange. The pressure may be adapted accordingly.

The compressed partly purified gas is contacted with absorbing liquidabsorbing liquid. The absorbing liquid comprises one or more amine compounds Suitable amine compounds are primary, secondary and tertiary amines. Preferably, the amines comprise at least one hydroxyalkyl moiety. The alkyl group in such moiety suitably comprises from 1 to 4 carbon atoms. In case of secondary and tertiary amines, the amine compounds preferably comprise one or more alkyl and hydroxyalkyl groups each with preferably from 1 to 4 carbon atoms. Suitable examples of amine compounds include monoethanol amine, monomethanol amine, monomethyl-ethanolamine, diethyl-monoethanolamine, diethanolamine, triethanolamine, di-isopropanolamine, diethyleneglycol monoamine, methyldiethanolamine and mixtures thereof. Other suitable compounds are N,N′-di(hydroxyalkyl)piperazine, N,N,N′,N′-tetrakis(hydroxyalkyl)-1,6-hexanediamine, in which the alkyl moiety may comprise from 1 to 4 carbon atoms.

The absorbing liquid may also comprise physical solvents. Suitable physical solvents include tetramethylene sulphone (sulpholane) and derivatives, amides of aliphatic carboxylic acids, N-alkyl pyrrolidone, in particular N-methylpyrrolidine, N-alkyl piperidones, in particular N-methyl piperidone, methanol, ethanol, ethylene glycol, polyethylene glycols, mono- or di(C₁-C₄)alkyl ethers of ethylene glycol or polyethylene glycols, suitably having a molecular weight from 50 to 800, and mixtures thereof.

The concentration of the amine compound in the absorbing liquid may vary within wide ranges. Advantageously, the absorbing liquid comprises at least 15% wt of water, from 10 to 65% wt, preferably from 30 to 55% wt of amine compounds and from 0 to 40% wt of physical solvent, all percentages based on the weight of water, amine compound and physical solvent.

The contacting step is suitably carried out at a temperature ranging from 15 to 90° C., preferably at a temperature of at least 20° C., more preferably from 25 to 80° C. As indicated above, the skilled person may choose any appropriate pressure for conducting the contacting step. Suitable pressures range from 10 to 150 bar, suitably from 20 to 100 bar. The compression step suitably raises the pressure by from 1 to 130 bar, preferably from 2 to 90 bar.

The contacting step results in purified hydrocarbon gas and absorbing liquid rich in acidic contaminants. In a preferred embodiment the absorbing liquid rich in acidic contaminants is regenerated. Such regeneration is known in the art. Suitable regeneration methods include flashing and/or stripping with an inert stripping gas. A very suitable stripping gas is steam, although other gases, such as nitrogen, may also be used. Thus regenerated absorbing liquid is suitably recycled to the contacting step.

The purified hydrocarbon gas obtainable by the process can be used as product. The recovered purified hydrocarbon gas may also be subjected to further treatment and/or purification. For instance, the purified hydrocarbon gas may be subjected to fractionation. In the event that the purified hydrocarbon gas is natural gas intended for pipeline transportation or for producing liquefied natural gas (LNG), in order to reach pipeline specifications or LNG specifications the purified natural gas may further purified. Further purification can for example be done in an additional cryogenic distillation column, suitably with a bottom temperature between −30 and 10° C., preferably between −10 and 5° C. A reboiler may be present to supply heat to the column. Suitably the top temperature column is between −110 and −80° C., preferably between −100 and −90° C. In the top of the cryogenic distillation column a condenser may be present, to provide reflux and a liquefied (LNG) product.

In the event that the hydrocarbon gas is natural gas, the purified natural gas can be processed further in known manners, for example by catalytic or non-catalytic combustion to produce synthesis gas, to generate electricity, heat or power, or for the production of liquefied natural gas (LNG), or for residential use. It is an advantage of the present process enables purification of natural gas comprising substantial amounts of acidic contaminants, resulting in purified natural gas comprising low levels of contaminants, especially of sulphur contaminants. The production of LNG from such natural gas, which would be very difficult if not impossible by conventional processes, is made possible. Thus, the invention also provides LNG obtained from liquefying purified natural gas obtained by the process. The LNG thus-obtained typically has very low concentrations of contaminants other than natural gas.

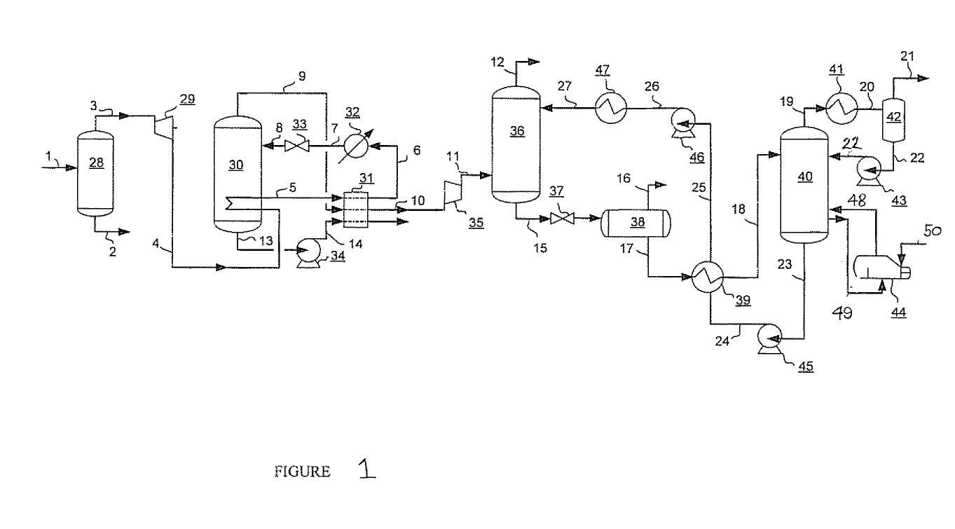

FIG. 1:

The present invention will be further illustrated by means of the following FIG. 1. In the description of the FIG. 1 reference is made to a natural gas stream as an example of the gas stream that may be treated in the process according to the present invention. The FIG. 1 shows a schematic flow scheme of an embodiment according to the invention.

A natural gas stream is introduced via a line 1 into a dehydrating unit 28. In the dehydration unit water is being removed from the natural gas stream, e.g., by means of molecular sieves. The water is eventually removed via a line 2. The dehydrated natural gas is passed via a line 3 to a turbo-expander 29 where it is expanded and cooled, and subsequently forwarded via a line 4. The line 4 is passed via a bundle coil, located in a vessel 30, from which it emerges as a line 5. In the vessel 30 the bundle coil acts as a heat exchanger for solid acidic contaminants that are collected in the bottom of vessel 30, thereby melting solid acidic contaminants. As described above, other embodiments are also possible. The natural gas in line 5 is cooled further. Via a heat exchanger 31 the natural gas stream is passed via a line 6 to a further optional heat exchanger 32. Via a line 7 the further cooled natural gas stream is passed to a Joule-Thomson valve 33 where it is expanded such that acidic contaminants solidify so that a slurry of acidic contaminants and liquid hydrocarbons pass through line 8 to the vessel 30, where the slurry falls down and from which partly purified gas is withdrawn at the top via a line 9. The FIGURE shows that short line 8 connects the Joule-Thomson valve with the vessel 30. This line is typically short so that blocking of the line by solids is prevented. It is also possible to do away with the line altogether and connect the Joule-Thomson valve directly to the wall of vessel 30.

The slurry in the bottom of vessel 30 is heated by the natural gas stream that flows through the bundle coil of line 4, thereby melting solid acidic contaminants. The bundle coil is just an example of a way to heat and melt the solid acidic contaminants. Other heating means are also possible. One may use an electric immersion heart, as suggested in WO-A 2007/030888. One may also add relatively warm natural gas liquids to the solid acidic contaminants, as suggested in WO-A 2004/070297. A preferred way is to heat at least part of the liquid that is withdrawn from the vessel 30 and recycle thus heated contaminants, which may be liquid or vaporous, into the vessel 30. Combinations of any of these heating means are also possible.

The line 13 from the bottom of the vessel 30 leads the melted contaminants to an optional pump 34, and via a line 14 and heat exchanger 31 the contaminants are withdrawn. In heat exchanger 29 the cold contaminants in line 14 and cold partly purified gas in line 9 are subjected to heat exchange with the natural gas stream in line 5. The streams are shown in co-current fashion. It is evident to the skilled person that the streams may also be arranged in a counter-current way, e.g., the relatively warm natural gas steam in counter-current with the two other streams. It will be appreciated that it is also feasible to use only one of the other streams or use a stream from another process, such as a stream from an LNG plant and/or an air separation plant.

From the heat exchanger 31 the partly purified gas is passed via a line 10 to a compressor 35. The compression energy for compressor 35 is suitably provided by the expander 29. The compressed gas may be passed by line 11 to a contacting vessel 36. Optionally, when higher pressures are desired, the compressed gas may first be brought to a still higher pressure by means of a second compressor.

In the contacting vessel 36 the compressed partly purified gas is contacted, preferably in counter-current with an absorbing liquid comprising an amine, e.g., methyldiethanol amine. The absorbing liquid is introduced into the vessel 36 via a line 27. The sweet hydrocarbon product is discharged via a line 12 at the top of the vessel 36 and a loaded absorbing liquid is withdrawn from the bottom via a line 15. Via a valve 37 via which the flow and liquid level in the vessel 36 may be controlled, the loaded solution is fed into a flashing vessel 38, where the pressure is released so that any hydrocarbons that are entrained and also part of the acidic contaminants can be withdrawn via a line 16. Such gas can be used as fuel gas or may be recycled to the gas in, e.g., line 11. Absorbing liquid that still contains significant amounts of acidic contaminants is discharged via a line 17. After heat exchange in an indirect heat exchanger 39, the absorbing liquid is passed via a line 18 into a stripping column 40. Steam, introduced into the column 40 via a line 48, strips acidic contaminants and a mixture of acidic contaminants and steam is withdrawn from the column via a line 19. Via a heat exchanger wherein the vapour is cooled and condensed and a line 20 the resulting mixture is passed to a separator 42 where the condensed water is separated from vaporous acidic contaminants. The acidic contaminants are recovered via a line 21 and the condensed water is recycled to the column 40 via a line 22 and an optional pump 43.

Near the bottom of the column 40 a liquid is withdrawn via a line 49 which is passed to a reboiler 44 to create steam that is used as stripping gas. If desired make up water may be provided via a line 50.

Regenerated absorbing liquid is discharged via a line 23, and via an optional pump 45 and a further line 24 used in the heat exchanger 39. The absorbing liquid leaves the heat exchanger 39 via a line 25 and pumped via an optional pump 46 to line 27. Dependent on the desired temperature the absorbing liquid may be heated or cooled, as desired, in an air or water cooler 47 before being introduced into the contacting vessel 36 via line 27.

1. A process for producing purified hydrocarbon gas from a gas stream comprising methane and acidic contaminants, which process comprises the steps:
(a) cooling the gas stream by expansion to form a mixture comprising solid and/or liquid acidic contaminants and a vapour containing gaseous hydrocarbons and a reduced amount of acidic contaminants;
(b) separating the solid and/or liquid acidic contaminants from the first mixture, yielding partly purified gas;
(c) compressing the partly purified gas; and
(d) contacting the compressed partly purified gas with an absorbing liquid to yield the purified hydrocarbon gas.

2. Process as claimed in claim 1 in which the gas stream is expanded from a pressure ranging from 20 to 200 bar to a pressure of 10 to 40 bar.

3. Process as claimed in claim 1, in which the gas stream is cooled to a temperature ranging from −40 to −100° C.

4. Process as claimed in claim 1, in which the gas stream is cooled to a temperature ranging from 1 to 40° C. above the freeze out temperature of the first acidic contaminant to freeze out, the freeze out temperature being the temperature at which solid contaminants are formed.

5. Process as claimed in claim 4, in which energy that is recovered at the expansion step or steps of the gas stream is used for the compression step or steps of the partly purified gas.

6. Process as claimed in claim 1, in which the absorbing liquid comprises a chemical solvent or a physical solvent or mixtures thereof.

7. Process as claimed in claim 1, in which the chemical solvent is selected from the group consisting of monoethanol amine, monomethanolamine, monomethyl-ethanolamine, diethyl-monoethanolamine, diethanolamine, triethanolamine, di-isopropanolamine, diethyleneglycol monoamine, methyldiethanolamine and mixtures thereof.

8. Process as claimed in claim 1, in which the physical solvent is selected from the group consisting of tetramethylene sulphone (sulpholane) and derivatives, amides of aliphatic carboxylic acids, N-alkyl pyrrolidone, in particular N-methylpyrrolidine, N-alkyl piperidones, in particular N-methyl piperidone, methanol, ethanol, ethylene glycol, polyethylene glycols, mono- or di(C₁-C₄)alkyl ethers of ethylene glycol or polyethylene glycols, suitably having a molecular weight from 50 to 800, and mixtures thereof.

9. Process as claimed in claim 1, in which step (d) is carried out at a temperature ranging from 15 to 90° C. and a pressure ranging from 10 to 150 bar.

10. Process as claimed in claim 1, in which step (d) results in purified hydrocarbon gas and absorbing liquid rich in acidic contaminants, which rich absorbing liquid is regenerated.

11. Process as claimed in claim 10, in which the regeneration is accomplished via flashing or stripping with an inert stripping gas.

12. Process as claimed in claim 1, in which the gas stream has been dehydrated.

13. Process as claimed in claim 1, wherein the purified gas is purified natural gas, the process further comprising the step of cooling the purified natural gas to obtain liquefied natural gas.
